(19) United States
(12) Patent Application Publication
(10) Pub. No.: US 2025/0322970 A1
(43) Pub. Date: Oct. 16, 2025

(54) FEDERATED ACCESS TO DATA RECORDS

(71) Applicant: Medosphere Inc., Tustin, CA (US)

(72) Inventors: Larry PHAM, Tustin, CA (US); Christopher Burton ONEIL, Tustin, CA (US); Taewook KANG, Tustin, CA (US)

(21) Appl. No.: 19/176,990

(22) Filed: Apr. 11, 2025

Related U.S. Application Data
(60) Provisional application No. 63/633,201, filed on Apr. 12, 2024.

(51) Int. Cl. G16H 80/00; G06F 21/32; G06V 40/16; G16H 10/60; H04N 23/66

(52) U.S. Cl. CPC G16H 80/00; G06F 21/32; G06V 40/172; G16H 10/60; H04N 23/66

(57) ABSTRACT

The present invention provides a secure system and method for sharing medical information among authorized medical professionals. The system ensures that only verified medical professionals can access the platform by requiring a medical license and utilizing facial recognition technology for authentication. The platform includes a content management component for uploading and accessing medical information, a digital rights manager to prevent unauthorized copying or removal of content, and a messaging system for secure communication between users. The system is designed to be HIPAA-compliant and includes features such as patient consent for video content, native voice triggers for camera movements during surgical procedures, and a customizable chat system for enhanced user experience. The invention aims to streamline the verification process, enhance security, and promote patient safety while facilitating the sharing of medical knowledge and techniques.

### CROSS-REFERENCE TO RELATED APPLICATIONS

This application claims the benefit of U.S. Provisional Application No. 63/633,201, filed on April 12, entitled “Federated Access to Data Records”. The entire disclosure of the provisional application is incorporated herein by reference.

### BACKGROUND

Medical professionals, such as doctors, play a vital role in providing healthcare services to patients. As such, it is important to establish a reliable and efficient means of verifying the identity and credentials of doctors to ensure that only qualified professionals are providing medical care. The National Provider Identifier (NPI) is a unique identification number that is assigned to healthcare providers in the United States.

Efforts to protect patient data under HIPAA, particularly electronic data, involve comprehensive strategies to ensure the confidentiality, integrity, and availability of sensitive information. The HIPAA Security Rule sets national standards for safeguarding electronic Protected Health Information (e-PHI). Healthcare providers, health plans, clearinghouses, and their business associates must implement administrative, physical, and technical safeguards. Administrative safeguards include policies and procedures that guide data protection efforts. Physical safeguards involve securing physical access to data storage and processing facilities. Technical safeguards encompass measures like encryption, secure storage methods, and multi-factor authentication to prevent unauthorized access. Regular risk assessments help identify vulnerabilities and ensure compliance with HIPAA regulations. By adopting these strategies, healthcare organizations can protect patient data, maintain trust, and ensure operational continuity.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present invention provides a secure system and method for sharing medical information among authorized medical professionals. The system ensures that only verified medical professionals can access the platform by requiring a medical license and utilizing facial recognition technology for authentication. The platform includes a content management component for uploading and accessing medical information, a digital rights manager to prevent unauthorized copying or removal of content, and a messaging system for secure communication between users. The system is designed to be HIPAA-compliant and includes features such as patient consent for video content, native voice triggers for camera movements during surgical procedures, and a customizable chat system for enhanced user experience. The invention aims to streamline the verification process, enhance security, and promote patient safety while facilitating the sharing of medical knowledge and techniques.

### BRIEF DESCRIPTION OF THE DRAWINGS

The technology described herein is illustrated by way of example and not limitation in the accompanying figures in which like reference numerals indicate similar elements and in which:

FIG. 1 is a diagram of a computing system suitable for implementations of the technology described herein;

FIG. 2 is a block diagram of an example operating environment for medical information sharing system, in accordance with an aspect of the technology described herein;

FIG. 3 is a home screen for a medical-information application, in accordance with an aspect of the technology described herein;

FIG. 4 is an account creation screen for a medical-information application, in accordance with an aspect of the technology described herein;

FIG. 5 is a password recovery screen for a medical-information application, in accordance with an aspect of the technology described herein;

FIG. 6 is a login screen for a medical-information application, in accordance with an aspect of the technology described herein;

FIG. 7 is a registration screen for a medical-information application, in accordance with an aspect of the technology described herein;

FIG. 8 is an email verification screen for a medical-information application, in accordance with an aspect of the technology described herein;

FIG. 9 is a license verification screen for a medical-information application, in accordance with an aspect of the technology described herein;

FIG. 10 is an identity verification screen for a medical-information application, in accordance with an aspect of the technology described herein;

FIG. 11 is a medical license verification screen for a medical-information application, in accordance with an aspect of the technology described herein;

FIG. 12 is a registration review screen for a medical-information application, in accordance with an aspect of the technology described herein;

FIG. 13 is a video view screen for a medical-information application, in accordance with an aspect of the technology described herein;

FIG. 14 is a medical professional network screen for a medical-information application, in accordance with an aspect of the technology described herein;

FIG. 15 is an instant message screen for a medical-information application, in accordance with an aspect of the technology described herein;

FIG. 16 is a profile screen for a medical-information application, in accordance with an aspect of the technology described herein;

FIG. 17 is a video gallery screen for a medical-information application, in accordance with an aspect of the technology described herein;

FIG. 18 is a video creation screen for a medical-information application, in accordance with an aspect of the technology described herein;

FIG. 19 is a video publication screen for a medical-information application, in accordance with an aspect of the technology described herein;

FIG. 20 is a video pull down screen for a medical-information application, in accordance with an aspect of the technology described herein;

FIG. 21 is a block diagram showing a computing device suitable for implementations of the technology described herein;

FIG. 22 is a block diagram showing flow chart of a method for sharing medical information suitable for implementations of the technology described herein;

FIG. 23 is a block diagram showing flow chart of a method for sharing medical information suitable for implementations of the technology described herein; and

FIG. 24 is a block diagram showing flow chart of a method for sharing medical information suitable for implementations of the technology described herein.

### DETAILED DESCRIPTION

The various technologies described herein are set forth with sufficient specificity to meet statutory requirements. However, the description itself is not intended to limit the scope of this patent. Rather, the inventors have contemplated that the claimed subject matter might also be embodied in other ways, to include different steps or combinations of steps like the ones described in this document, in conjunction with other present or future technologies. Moreover, although the terms “step” and/or “block” may be used herein to connote different elements of methods employed, the terms should not be interpreted as implying any order among or between various steps herein disclosed unless and except when the order of individual steps is explicitly described.

The present invention provides a secure system and method for sharing medical information among authorized medical professionals. The system ensures that only verified medical professionals can access the platform by requiring a medical license and utilizing facial recognition technology for authentication. The platform includes a content management component for uploading and accessing medical information, a digital rights manager to prevent unauthorized copying or removal of content, and a messaging system for secure communication between users. The system is designed to be HIPAA-compliant and includes features such as patient consent for video content, native voice triggers for camera movements during surgical procedures, and a customizable chat system for enhanced user experience. The invention aims to streamline the verification process, enhance security, and promote patient safety while facilitating the sharing of medical knowledge and techniques.

The present invention provides a novel Doctor Verification Method that employs both National Provider Identifier (NPI) and photographs of the doctor alongside their state-issued medical license. The use of NPI, along with other identification methods, can help to streamline the verification process and enhance the security and reliability of the authentication process. The system utilizes advanced image recognition and verification algorithms to compare the photograph of the doctor with the photograph on their state-issued medical license. This method ensures a secure and reliable means of verifying the identity and credentials of medical professionals, with applications in healthcare institutions, insurance companies, and other relevant sectors. The present invention thus facilitates streamlined verification procedures and promotes patient safety.

The technology described herein is a HIPAA-compliant system and method designed for the generation and distribution of medical content, including medical video content. This medical content is made available through streaming or video on demand (VOD) for the purposes of medical education, all while ensuring the privacy and rights of the patient. The system utilizes a knowledge-based verification process to confirm the identity of individuals as medical professionals before allowing video capture and requires patient consent prior to generating, broadcasting, or publishing any footage. To maintain HIPAA compliance, the system employs video authentication and encryption methods before the content is distributed or consumed on authorized devices.

The copyright, distribution, licensing and optional monetization of medical content, including educational materials and procedural techniques leverages Digital Millennium Copyright Act (DMCA) whereby Providers can register any content containing PHI for the purpose of obtaining copyright protection and licensing under various modalities. Licensing options include open licenses (e.g. Creative Commons), proprietary licenses and royalty-based licenses. The content is assigned a unique digital watermark or fingerprint for copyright enforcement, licensing administration and royalty calculation under DMCA. The watermarked content can then be securely distributed and shared under the license terms. The system tracks sharing, modifications, performance and usage of the content to detect copyright infringement, license violations or calculate royalties.

The technology allows healthcare providers to maintain ownership and control over their medical works, that they first generate and create, while still complying with privacy laws regarding patient health information and only using PHI for which informed consent has been obtained. Providers can license proprietary medical techniques under flexible terms and get compensation for their intellectual property through royalties, all within the scope of patients' consent.

This system thus facilitates an exchange for medical knowledge and techniques, allowing healthcare providers to more easily obtain intellectual property rights, distribute and license or sell their medical creations, and get remuneration through royalties or other licensing options. Patient privacy remains protected by de-identification and consent requirements on the use of personal health information. The technology catalyzes medical innovation and benefits both healthcare providers and patients.

The technology described herein may provide video creation technology, such as an automated camera tracking system to help generate high quality surgery videos. The present technology relates to an innovative automated camera tracking system designed to provide hands-free operation during surgical procedures. The system incorporates facial recognition technology, native voice triggers defined by the movie script integration to efficiently track the movement of the doctor or patient and adjusting the camera's position accordingly to ensure optimal video capture. Traditional camera systems used in surgical settings often require manual adjustments or pre-programmed controls, which can be impractical in the dynamic environment of surgery. The interactive camera system described herein can autonomously adjust to the requirements of the surgery in real-time.

The system combines facial recognition technology, native voice triggers, and movie script integration to provide a comprehensive solution for hands-free camera operation during surgery. Facial recognition technology accurately identifies and tracks the faces of the doctor and the patient, allowing the camera to automatically adjust its position to maintain focus on the selected individual. Native voice triggers enable the doctor to use pre-defined voice commands to initiate specific camera movements, such as panning, tilting, or zooming. Movie script integration allows the doctor to create a detailed script that defines both the narration and the desired camera angles and zoom levels. The system then utilizes these inputs to trigger the appropriate camera actions in sync with the doctor's narration during the surgery.

The technology described herein provides for a HIPAA compliant chat system that enables doctors to communicate with one another before, during, and after medical procedures, such as surgical operations. The system includes features that ensure only verified doctors can access the platform and that patient consent has been given prior to any medical conversations taking place between them. Furthermore, it also allows for educational resources to be shared amongst medical professionals, facilitating the furthering of knowledge in the field. The chat system is a secure and convenient way for doctors to communicate with one another during medical procedures while remaining HIPAA compliant. The system includes may include an encryption mechanism may be used to protect all communications and data shared on the chat system, ensuring that patient information remains confidential and secure. The system includes a user-friendly interface that enables doctors to easily communicate with each other during medical procedures, regardless of their physical location. The system includes an archive feature that securely stores all communications on the platform, allowing doctors to reference past conversations and shared resources for future use.

The message platform may be integrated with other medical systems and software to allow for seamless sharing of patient information and medical records between doctors, as needed and with appropriate consent. A customizable setting allows doctors to tailor the chat system to their individual preferences and needs, enhancing the user experience and efficiency of the platform.

As used herein, medical information may be any information, including genetic information, whether oral or recorded in any form or medium, that is created or received by a health care provider, health plan, public health authority, employer, life insurer, school or university, or health care clearinghouse and relates to the past, present, or future physical or mental health or condition of an individual and/or the provision of health care to an individual.

As used herein, a medical professional, also referred to as a health care provider, is a Doctor of Medicine or osteopathy who is authorized to practice medicine or surgery (as appropriate) by the State in which the medical professional practices. Additional examples of health care providers may include, but are not limited to, podiatrists, dentists, clinical psychologists, optometrists, chiropractors, nurse professionals, nurse-midwives, clinical social workers, and physician assistants who are authorized to practice under State law and who are performing within the scope of their practice as defined under State law.

Having briefly described an overview of aspects of the technology described herein, an operating environment in which aspects of the technology described herein may be implemented is described below to provide a general context for various aspects.

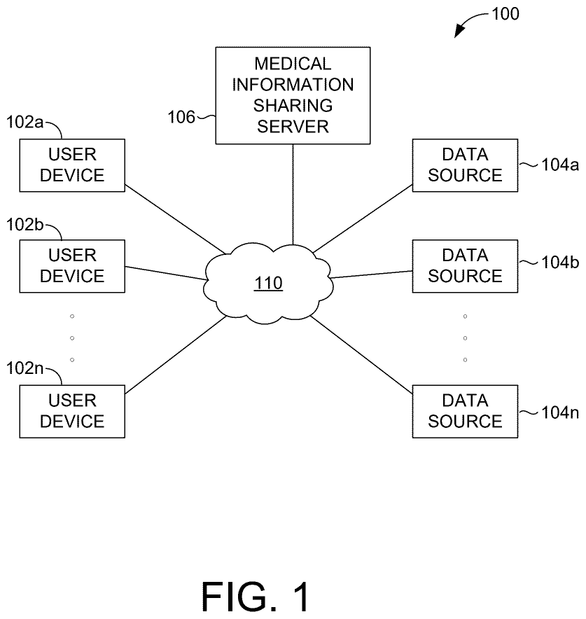

Turning now to FIG. 1, a block diagram is provided showing an example operating environment 100 in which some embodiments of the present disclosure can be employed. This and other arrangements described herein are set forth only as examples. Other arrangements and elements (for example, machines, interfaces, functions, orders, and groupings of functions) can be used in addition to or instead of those shown, and some elements can be omitted altogether for the sake of clarity. Further, many of the elements described herein are functional entities that are implemented as discrete or distributed components or in conjunction with other components, and in any suitable combination and location. Various functions described herein as being performed by one or more entities are carried out by hardware, firmware, and/or software. For instance, some functions are carried out by a processor executing instructions stored in memory.

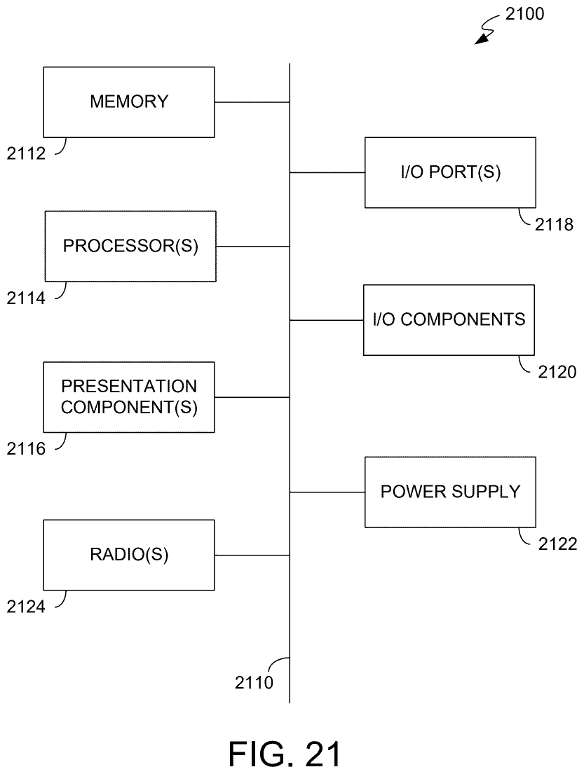

Among other components not shown, example operating environment 100 includes several user computing devices, such as user devices 102a through 102n; several data sources, such as data sources 104a and 104b through 104n; medical information sharing server 106; and network 110. Each of the components shown in FIG. 1 is implemented via any type of computing device, such as computing device 2100 illustrated in FIG. 21, for example. In one embodiment, these components communicate with each other via network 110, which includes, without limitation, one or more local area networks (LANs) and/or wide area networks (WANs). In one example, network 110 comprises the internet, intranet, and/or a cellular network, amongst any of a variety of possible public and/or private networks.

Any number of user devices, servers, and data sources can be employed within operating environment 100 within the scope of the present disclosure. Each may comprise a single device or multiple devices cooperating in a distributed environment, such as the distributed computing device 2100 in FIG. 21. For instance, medical information sharing server 106 may be provided via multiple devices arranged in a distributed environment that collectively provides the functionality described herein. Additionally, other components not shown may also be included within the distributed environment.

User devices 102a through 102n can be client user devices on the client-side of operating environment 100, while medical information sharing server 106 can be on the server-side of operating environment 100. Medical information sharing server 106 can comprise server-side software designed to work in conjunction with client-side software on user devices 102a through 102n to implement any combination of the features and functionalities discussed in the present disclosure. In one aspect, the medical information sharing server 106 hosts a medical-information sharing system. In aspects, the user devices 102a through 102n provide a user interface to the medical-information sharing system. This division of operating environment 100 is provided to illustrate one example of a suitable environment, and there is no requirement for each implementation that any combination of medical information sharing server 106 and user devices and 102a through 102n remain as separate entities.

In some embodiments, user devices 102a through 102n comprise any type of computing device capable of use by a user. For example, in one embodiment, user devices 102a through 102n are the type of computing device 2100 described in relation to FIG. 21. By way of example and not limitation, a user device is embodied as a personal computer (PC), a laptop computer, a mobile device, a smartphone, a tablet computer, a virtual-reality (VR) or augmented-reality (AR) device or headset, a handheld communication device, an embedded system controller, a consumer electronic device, a workstation, any other suitable computer device, or any combination of these delineated devices.

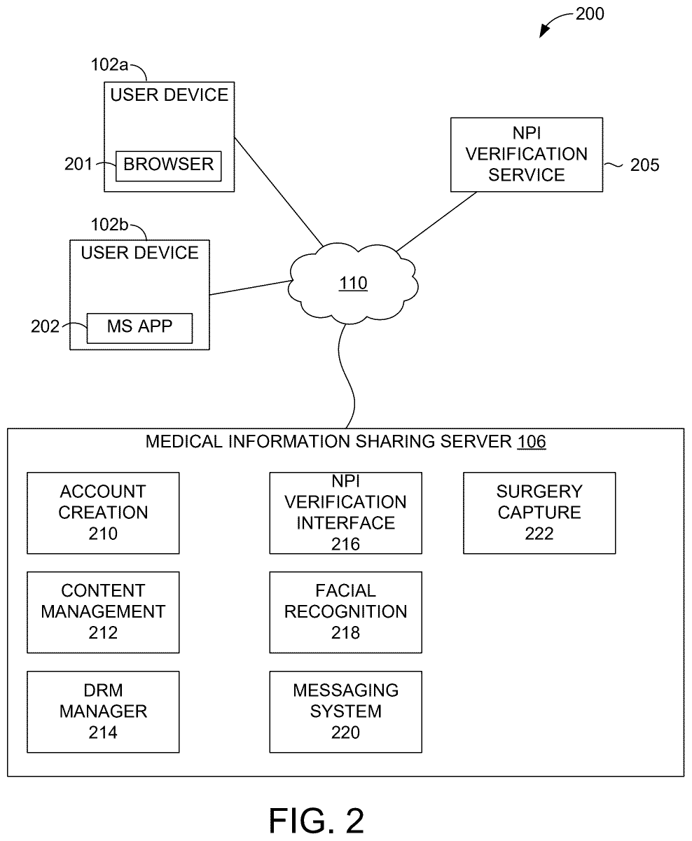

In some embodiments, data sources 104a and 104b through 104n comprise data sources and/or data systems, which are configured to make data available to any of the various constituents of operating environment 100 or system 200 described in connection to FIG. 2. The data sources may include information and/or services used to confirm identity, including a user's status as a medical professional. For example, the data sources 104a and 104b through 104n may include user profiles for different medical professionals. The data sources 104a and 104b through 104n can also store medial videos, medical chats, and the like. Certain data sources 104a and 104b through 104n are discrete from user devices 102a through 102n and medical information sharing server 106 or are incorporated and/or integrated into at least one of those components. In one embodiment, one or more of data sources 104a and 104b through 104n comprise one or more sensors, which are integrated into or associated with one or more of the user device(s) 102a through 102n or medical information sharing server 106. For example, the data sources could include a web camera used to capture a medical operation or video chat.

Operating environment 100 can be utilized to implement one or more of the components of system 200, as described in FIG. 2. Operating environment 100 can also be utilized for implementing various methods.

Referring now to FIG. 2 with FIG. 1, a block diagram is provided showing aspects of an example computing system architecture suitable for implementing some embodiments of the disclosure and designated generally as system 200. FIG. 2 illustrates one or more components of a medical-information sharing application.

The system 200 represents only one example of a suitable computing system architecture. Other arrangements and elements can be used in addition to or instead of those shown, and some elements may be omitted altogether for the sake of clarity. Further, as with operating environment 100, many of the elements described herein are functional entities that may be implemented as discrete or distributed components or in conjunction with other components, and in any suitable combination and location. These components may be embodied as a set of compiled computer instructions or functions, program modules, computer software services, or an arrangement of processes carried out on one or more computer systems.

In one embodiment, the functions performed by components of system 200 are associated with a medical-information sharing application. These components, functions performed by these components, and/or services carried out by these components may be implemented at appropriate abstraction layer(s) such as the operating system layer, application layer, and/or hardware layer of the computing system(s). Alternatively, or in addition, the functionality of these components, and/or the embodiments described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs). Additionally, although functionality is described herein with regards to specific components shown in example system 200, it is contemplated that in some embodiments functionality of these components can be shared or distributed across other components and/or computer systems.

The system 200 includes a first user device 102a with a browser 201 used to access the medical information sharing server 106 and a second user device 102b with a medical information sharing application 202 used to access the medical information sharing server 106. The NPI verification service 205 may be used to verify that a potential user has a medical license. The medical information sharing server 106 includes an account creation component 210, a content management component 212, a digital rights manager 214, an NPI verification interface 216, a facial recognition component 218, and a messaging system 220.

The first user device 102a includes a browser 201 used to access the medical information sharing server 106. In an aspect, a browser may be used to securely access medical information after undergoing an authorization process for the user of the browser, which may include submitting a username, password, and facial image. When a web browser accesses a secure content provider service, it typically follows these various processes. Initially, the user, through their web browser (also known as the user agent), requests a web resource protected by a secure content provider service. The browser uses HTTPS (HyperText Transfer Protocol Secure) to ensure secure communication with the web server. HTTPS encrypts every data packet sent using either SSL (Secure Sockets Layer) or TLS (Transport Layer Security) encryption. This encryption ensures that any information the user enters into a site is sent in a secure manner and cannot be easily intercepted by hackers.

The secure medical information sharing service, wishing to know the identity of the user, issues an authentication request to an identity provider through the user agent. This may be done through a process called Single Sign-On (SSO), which allows a user to log in with a single ID to any of several related, yet independent, software systems. The identity provider verifies the user's credentials and sends back an authentication token to the secure content provider service. This token is used to establish the user's identity and access rights. Once the user is authenticated and authorized, they can access the secure medical information sharing server 106. This process ensures that only authorized users can access the secure content, thereby protecting the content from unauthorized use and piracy.

The second user device 102b includes a medical information sharing application 202 used to access the medical information sharing server 106. In an aspect, the medical information sharing application is a smartphone application. The medical information sharing application 202 may interact with a back-end service in various ways. The medical information sharing application 202 sends a request to the back-end service, such as the medical information sharing server 106. This request could be for data retrieval, data update, or any other service that the medical information sharing server 106 provides. For example, a user could view an existing video or upload a new video through the application. The medical information sharing server 106 processes the request. This could involve querying a database, performing some computations, or interacting with other services. After processing the request, the medical information sharing server 106 sends a response back to the medical information sharing application 202. This response contains the result of the request, which could be data, a confirmation of a successful update, an error message, etc. The medical information sharing application 202 may perform data synchronization across platforms, as well as data storage capabilities. It requires the ability to send notification messages and the capacity to support different HTTP methods. For example, the medical information sharing application 202 may receive updated instant messages, comments, and the like.

The medical information sharing application 202 may provide valid credentials to the medical information sharing server 106, possibly in the form of an authentication token. This token may be included in the header of the HTTP request. The medical information sharing application 202 may use facial recognition as part of authentication. Based on the response from the medical information sharing server 106, the application updates its user interface. For example, it could display the retrieved data, confirm the success of an update, or show an error message. The retrieved data could include medical information, such as a video showing a surgery.

The National Provider Identifier (NPI) verification service 205 may be used to verify that a potential user has a medical license. The NPI is a unique 10-digit identification number issued to health care providers in the United States by the Centers for Medicare and Medicaid Services (CMS). The NPI has replaced the Unique Physician Identification Number (UPIN) as the required identifier for Medicare services, and is used by other payers, including commercial healthcare insurers. The NPI is publicly accessible. In aspects of the technology described herein, a user's NPI information is confirmed as part of account creation process performed by the account creation component 210.

The account creation component 210 facilitates account creation and management. An account may be created by generating a unique username and password for a medical professional. The account creation component 210 may request and confirm that a potential account holder has a medical license by using the NPI verification. In aspects, the account creation component 210 generates a facial identity of the account holder. The facial identity may be compared to a picture on a driver license, passport, or other form of reliable identification. To authorize a facial identity for association with a user, a “live” image provided through a camera associated with the user device (e.g., 102a or 102b) may need to match the image on the reliable ID.

The content management component 212 manages content upload and access. The content management system 212 may take the form of a Cloud-Based Content Management System (CMS) for videos, which is a software application that helps organizations store, organize, manage, and present online video content. It allows users to build an intricate, scalable video ecosystem without any programming knowledge. The content management system 212 may provide dedicated storage for large files and perform video compression.

The content management system 212 may provide disruption-free streaming. The CMS ensures that viewers can engage with the content across all devices, including mobile, without disruption. The content management system 212 allows arrangement of the video content to make it more accessible and presentable. Users may use video CMS software to analyze data, such as the total views on their content. As the content library expands, video CMS scales to accommodate the content growth. Example CMS systems that may be used include VPlayed, Dacast, Panopto, Brightcove, Wistia, IBM Cloud, Vimeo, Zype, Kaltura, and CMS Hub

The digital rights manager 214 applies DRM technology to the uploaded content to prevent copying or removal from the service. Digital Rights Management (DRM) is a technology used to protect digital content, including videos, from unauthorized use and piracy. DRM may use different encryption methods. DRM requires the video content to be stored and transmitted in an encrypted form. This means that the data is transformed by the digital rights manager 214 into a code that can only be accessed by those who have a decryption key. The encrypted video content must be packaged, often using multiple DRM schemes for greater device compatibility. The packaging process involves putting the video content into a format that is compatible with the DRM systems, generally MPEG-DASH or HLS (HTTP Live Streaming). When a user attempts to play back a video, the video player requests a key from a license server. The license server determines whether the user and device are authorized before issuing a license response with a decryption key. If the user is authorized, the player can then decrypt and play back the content for the user.

This process ensures that only authorized users and devices can play the video, thereby preventing the video from being copied or downloaded without permission. It's important to note that enabling DRM requires changes to at least three components of the streaming workflow: the content, the player, and the license server. There are many DRM systems available to protect video content. The most popular ones are Google's Widevine, Apple's FairPlay, and Microsoft's PlayReady. Each of these DRM systems is compatible with different web browsers, devices, and set-top boxes.

The NPI verification interface 216 validates the holders of a medical license by interacting with the NPI database. In an aspect, a medical license is provided with a name, address, and other information about a medical professional. The NPI verification interface 216 uses this information to confirm it matches information in the NPI database.

The facial recognition component 218 authenticates users with facial recognition. Facial recognition identifies and verifies individuals based on their unique facial features. It has gained prominence as a secure authentication method in various applications, including smartphones, access control systems, and surveillance cameras. Facial recognition falls under the broader category of biometric authentication. Unlike traditional security methods like PINs or passwords, which can be forgotten or stolen, biometrics rely on physical traits unique to each person. By analyzing facial characteristics, the system creates a digital representation (a template) of the individual's face. This may occur during the user registration process and subsequent access process.

During enrollment, the user captures several images of their face from different angles with a smart phone camera, digital camera, web camera, or the like. The system extracts key facial features, such as the distance between eyes, nose shape, and mouth contours. These features are used to create a facial template, a mathematical representation stored securely on the device or server. In aspects, the user also provides a driver license, passport, or other reliable identification including a picture of the user. During enrollment, the picture of the identification may be compared with the images provided to determine that they match within a threshold. The enrollment may be rejected if the live images do not match the image on the provided identification.

When the user attempts to access the medical information sharing service in the future, which is a secure area, the facial recognition system activates. A camera associated with the user device, such as the front-facing camera on a smart phone, captures a live image of the user's face. The system then compares this real-time image with the stored template. If the features match within a predefined threshold, access is granted.

Facial recognition technology involves several steps to ensure secure access to the medical information sharing service. Initially, the system captures an image or video frame containing one or more faces using a camera. Advanced algorithms detect the presence of faces by identifying key facial features such as the eyes, nose, and mouth. Once detected, the faces are aligned to ensure consistency in orientation and scale, making them easier to analyze. The system then extracts unique facial features from the aligned face, measuring distances between key landmarks and creating a mathematical representation of the face. These features are encoded into a digital format, often as a vector of numerical values, representing the unique characteristics of the face. The encoded face is then compared against a database of stored facial encodings, with matching algorithms determining the similarity between the input face and stored faces to identify potential matches. Based on the matching results, the system decides whether the face belongs to an authorized user and grants access if a match is found within a predefined threshold.

In practical application, users initially enroll by providing their facial data, capturing multiple images under different conditions to create a robust facial encoding. When a user attempts to access the medical information sharing service, the system captures a live image or video frame of their face and processes it through the facial recognition steps to verify the user's identity. If the system successfully verifies the user's identity, it grants access to the requested computer resources, such as logging into the medical information sharing service. For enhanced security, the system may continuously monitor the user's face during the session to ensure the same person remains in front of the device. This detailed process ensures secure and efficient access control using facial recognition technology.

Facial recognition technology can be used to compare a live image with the image on a government-issued ID to verify a person's identity. The process begins with the user capturing a clear photograph of their government-issued ID and taking a real-time image, also described as a live image. Advanced algorithms then analyze the ID, pinpointing facial features and scrutinizing text details to ensure the ID's authenticity. The system extracts the facial image from the ID and compares it with the live image using biometric facial matching algorithms. These algorithms assess the similarity between the two images by examining unique facial characteristics such as the distance between the eyes, the shape of the nose, and the contours of the mouth

To enhance security, the system employs passive liveness detection, which verifies that the live image is not a photo, video playback, or mask, ensuring the genuine presence of a real person. If the facial features match within a predefined threshold, the system confirms the user's identity and grants access to the requested resources.

The facial recognition system may accurately identify faces, even in less-than-ideal conditions, including low light or direct sunlight. The system may recognize faces even when tilted, turned, or expressing emotions. To prevent unauthorized access using photographs or videos, modern systems incorporate anti-spoofing techniques. Unlike passwords, which can be shared or stolen, a face recognition confirms who is accessing the content or service. The facial recognition may be one factor in a biometric Multi-Factor Authentication process. In aspects, the system may combine facial recognition with other factors (like fingerprint or voice) for stronger security. As previously mentioned, the user will also provide a username and password.

The messaging system 220 enables users to communicate with each other, with groups, and/or comment on content. Initially, the sender creates a message on their device and enters the recipient's identifier (like a phone number or username). The sender's device sends the message to a centralized server, often called a Message Service Center (SMSC) for SMS or a similar entity for other types of messages, which is part of the messaging system 220.

The messaging system 220 checks the recipient's identifier and determines the appropriate network or service to deliver the message. The server then sends the message to the recipient's network or service. If the recipient's device is not available (for example, if it's turned off or out of range), the message is stored on the server until the device becomes available. This is known as a “store-and-forward” service. Once the recipient's device is reachable, the server sends a notification to the device indicating that a new message is available. The recipient's device connects to the server to retrieve the message. The recipient's device receives the message and displays it to the recipient. The recipient's device may send a delivery confirmation back to the sender's server, indicating that the message was successfully received.

In terms of storage, messages and media files can be made accessible. The choice of database for storing these messages depends on the use cases, what's important to the users, and what the development team is familiar with. Messages can be retained indefinitely on the system, or they can be exported to an archive if the carrier chooses. This process ensures that messages are delivered reliably and can be retrieved even if the recipient's device was not available at the time the message was sent. It's important to note that this is a simplified explanation, and actual implementations can vary based on the specific technology and service provider. The messages may be associated with metadata linking them to content, topics of interest, or other categories. The metadata may be used surface and filter messages.

Message settings in the messaging system 220 offer a range of customization options to enhance user experience and privacy. Users can adjust notification preferences, such as choosing to be alerted with sounds, vibrations, or reminders when a new message arrives. Additionally, settings allow users to change the default messaging app, modify font and display sizes, and manage how multimedia files are sent and received. Privacy settings are crucial, enabling users to block messages from specific phone numbers, user ids, or groups, report spam, and turn off link previews for videos. Regarding visibility, the messaging system 220 may provide controls to limit who can view published messages. Individual settings allow users to restrict message visibility to specific contacts or groups, ensuring that only intended recipients can access the content. Platform settings may include options to share messages across different devices or platforms, while group settings enable users to manage who can participate in group conversations and view shared messages. These comprehensive settings ensure that users have control over their messaging experience, maintaining privacy and security across various contexts. If messages are associated with a specific patient, then the patient settings may govern. For example, the messages may only be viewing by a designated group, regardless of the settings associated with the medical professional posting the message. These same settings and preferences may apply to any post with the medical information sharing service, including videos.

The surgery capture component 222 helps create content. The surgery capture component 222 may provide video creation technology, such as an automated camera tracking system to help generate high quality surgery videos. The present technology relates to an innovative automated camera tracking system designed to provide hands-free operation during surgical procedures. The system incorporates facial recognition technology, native voice triggers defined by the movie script integration to efficiently track the movement of the doctor or patient and adjusting the camera's position accordingly to ensure optimal video capture. Traditional camera systems used in surgical settings often require manual adjustments or pre-programmed controls, which can be impractical in the dynamic environment of surgery. The need for a more sophisticated and interactive camera system that can autonomously adjust to the requirements of the surgery in real-time is evident.

The surgery capture component 222 may combine facial recognition technology, native voice triggers, and movie script integration to provide a comprehensive solution for hands-free camera operation during surgery. Facial recognition technology accurately identifies and tracks the faces of the doctor and the patient, allowing the camera to automatically adjust its position to maintain focus on the selected individual. Native voice triggers enable the doctor to use pre-defined voice commands to initiate specific camera movements, such as panning, tilting, or zooming. Native voice triggers are specific phrases or keywords that activate voice-controlled applications. These triggers may signal the start or end of an interaction between the user and the device. Voice triggers are designed to be recognized accurately and efficiently, even in noisy environments. They often involve multiple stages of processing to ensure privacy, reduce latency, and minimize false triggers. This includes distinguishing the device's primary user from other speakers and rejecting background noise.

Movie script integration allows the doctor to create a detailed script that defines both the narration and the desired camera angles and zoom levels. During filming, scripts guide the actions of automated cameras and/or drones. These devices follow pre-programmed movements and angles based on the script, ensuring consistent and precise shots. The transition from one shot to the next may be guided by voice triggers. The system then utilizes these inputs to trigger the appropriate camera actions in sync with the doctor's narration during the surgery.

The surgery capture component 222 may comprises the following components: a Facial recognition module that utilizes artificial intelligence and machine learning algorithms to identify and track the faces of the doctor and the patient, a voice command module that employs natural language processing algorithms to recognize specific native voice triggers and initiate corresponding camera movements, a movie script integration module that processes the doctor's script to synchronize camera actions with the narration, a camera control module that the camera's movements, including panning, tilting, and zooming, based on the input from the voice command and movie script integration modules, and a storage module that stores facial recognition data, voice command data, movie scripts, and other relevant information.

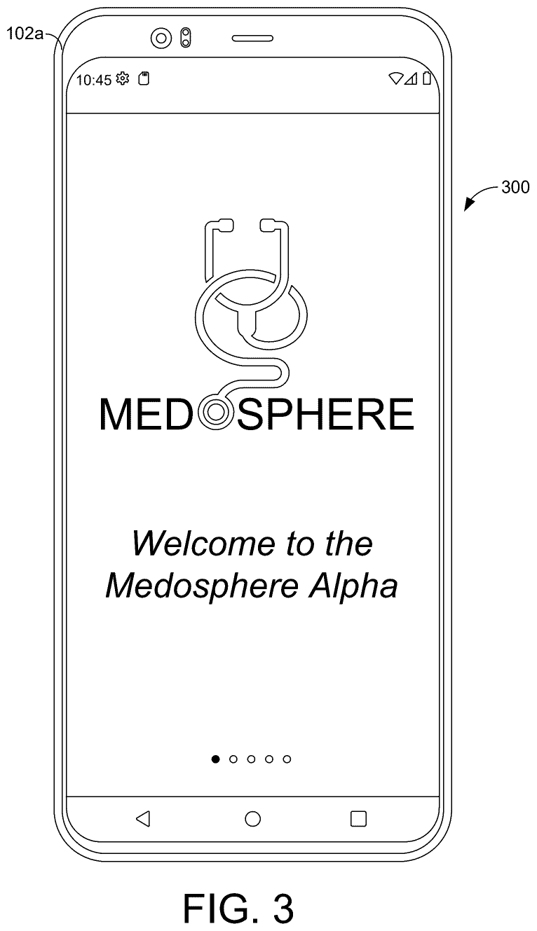

Turning now to FIG. 3, a home screen 300 for a medical information sharing service is shown, in accordance with an aspect of the technology described herein. The home screen 300 is displayed on a user device 102a. The home screen 300 may be part of a medical information sharing service designed to allow medical professionals to share relevant medical content with other medical professionals. The medical content can include videos, images, textual descriptions, analytical data, MRI data, x-ray data, ultrasound data, messages, and the like. The medical content can be shared for the purpose of receiving medical consultation and/or medical education, among other reasons. The user device 102a is depicted as a smart phone in FIGS. 3-20, however, the embodiments of the technology described herein are not limited to use on a smart phone. The technology may be deployed on any appropriate user device.

The home screen 300 may be the screen displayed upon first opening a medical information sharing service application running on the user device. Alternatively, the home screen 300 may be the screen displayed upon navigating to a webpage viewed through a web browser. In an aspect, the home screen 300 may be a default home screen. Once a user has an account set up, the user may select a custom home screen or a custom home screen may be selected automatically for the user. For example, the screen most frequently viewed by the user may be automatically set as the default homepage. In an aspect, the user may view different screens by swiping right or left on the home screen 300. In an aspect, additional views may be accessed through a drop-down menu (not shown) or selectable icons (not shown).

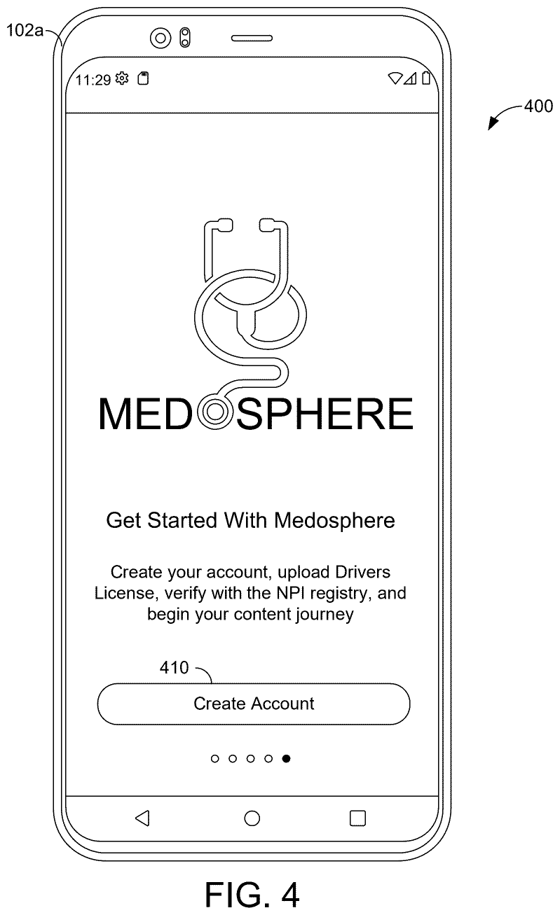

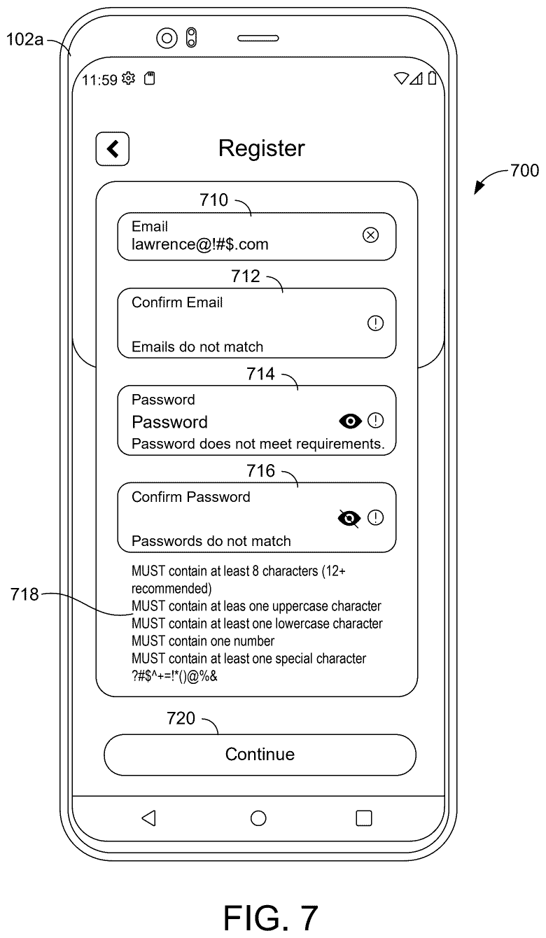

Turning now to FIG. 4, an account creation screen 400 for a medical information sharing service is shown, in accordance with an aspect of the technology described herein. The account creation screen 400 is displayed on a user device 102a. The account creation screen 400 allows the user to begin the account creation process by selecting the create account button 410. In an aspect, selecting the create account button 410 brings the user to the registration screen 700 shown in FIG. 7.

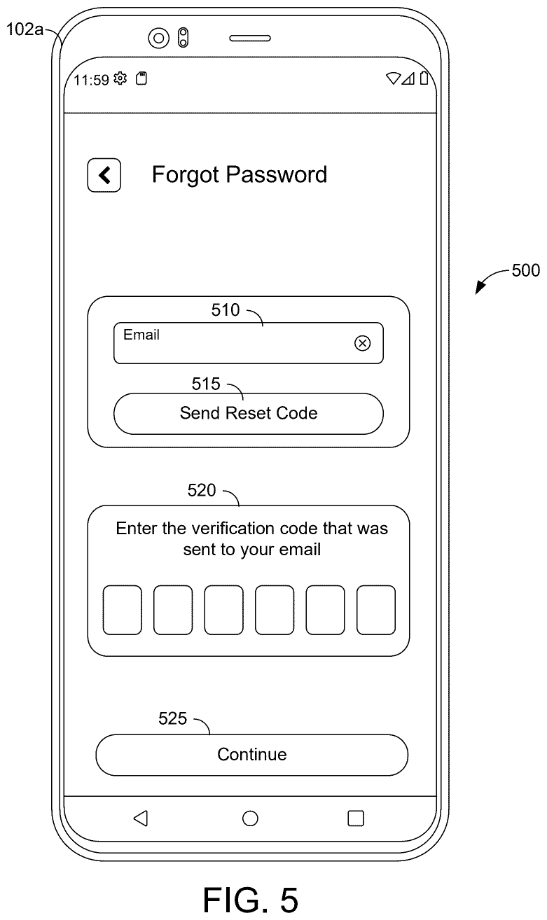

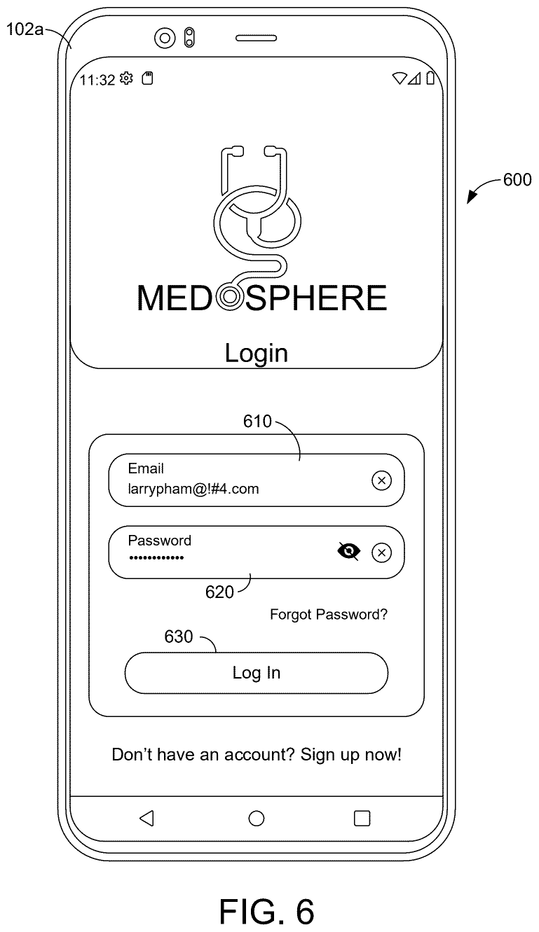

Turning now to FIG. 5, a password recovery screen 500 for a medical information sharing service is shown, in accordance with an aspect of the technology described herein. The password recovery screen 500 is displayed on a user device 102a. If the user already has an account, the user may login with a username and password. The password recovery screen 500 allows the user to recover their existing password by entering their email in the email box 510 and pushing the send reset code 515 button. The user may then be sent a reset code via email or some other means such as text message. In alternative embodiments, an authentication application may be used to provide the verification code. Upon receiving the verification code, the user may enter the verification code in the code box 520 and select the continue button 525. This may bring the user to an interface through which a new password may be selected. The user may then login using their new password as shown in FIG. 6.

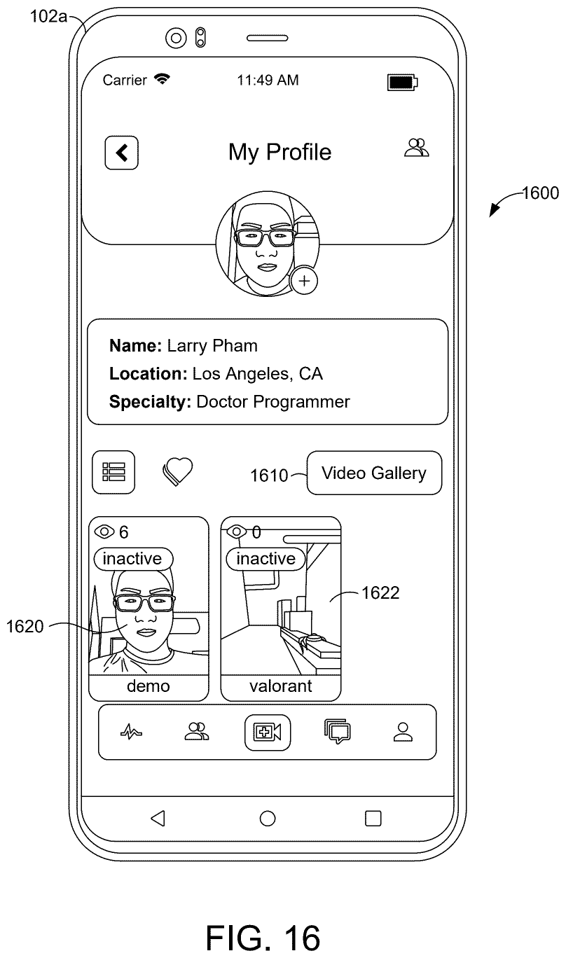

Turning now to FIG. 6, a login screen 600 for a medical information sharing service is shown, in accordance with an aspect of the technology described herein. The login screen 600 is displayed on a user device 102a. The login screen 600 allows the user to enter their email 610 in the email box and password in the password box 620. In this example, the email may serve as a username. In aspects, an identifier other than a person's email may be used as a username. Once the email and password are entered the user may select the login 630 button to initiate the login process. The login process may include receiving a live image of the user through a camera associated with user device 102a and comparing it to a stored image of the user. The user may be asked to position themselves (or the camera) in way that the user's face is visible and within an appropriate distance from the camera. Upon logging in, the user may be taken to a secured page, such as the profile page 1600 shown in FIG. 16.

Turning now to FIG. 7, a registration screen 700 for a medical information sharing service is shown, in accordance with an aspect of the technology described herein. The registration screen 700 is displayed on a user device 102a. The registration screen 700 allows the user to create a new account. The user may begin by entering their email address in the email box 710. To detect any potential typos in the email just entered, the user may be asked to confirm their email address by typing their email address a second time in the email confirmation box 712. If the two email addresses do not match, then the user may be asked to reenter their email address. If the email addresses match, then the user may be asked to create a password by typing a new password in the password box 714. The password may need to meet certain password strength requirements 718. The user may be alerted when the proposed password does not match the requirements. The user may continue to enter passwords until submitting one that matches the requirements. Once requirements are met, to detect any potential typos, the user may enter the same password in the confirmation password box 716. Once a password meeting the requirements is submitted, the password may be associated with the user account and subsequently used to access the account in combination with the username and/or email and facial recognition. The user may press the continue button 720 to access next steps of the registration process.

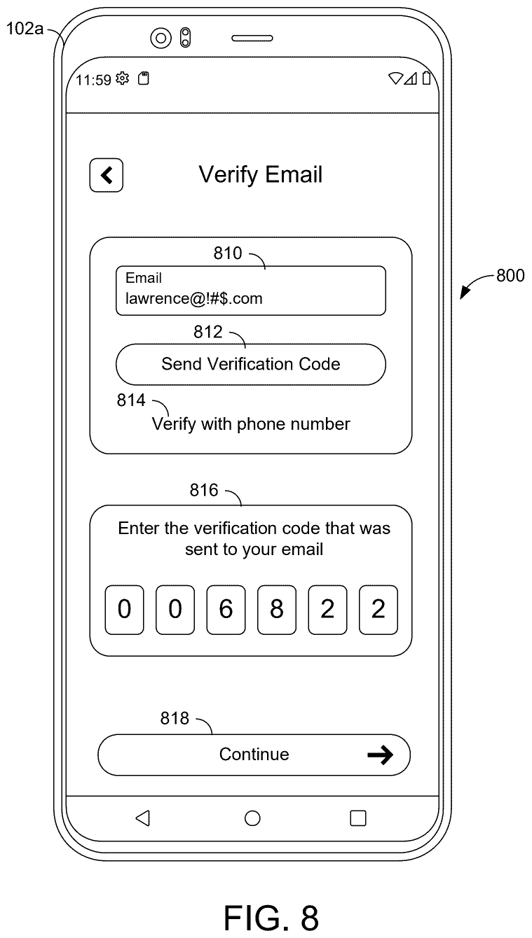

Turning now to FIG. 8, an email verification screen 800 for a medical information sharing service is shown, in accordance with an aspect of the technology described herein. The email verification screen 800 is displayed on a user device 102a. The email verification screen 800 allows the user to verify their email. The user's email address may be entered or displayed in the email address box 810. Upon pushing the send verification code button 812, the user may then be sent a verification code via email or some other means, such as text message if the user selects the phone mode button 814. In alternative embodiments, an authentication application may be used to provide the verification code. Upon receiving the verification code, the user may enter the verification code in the code box 816 and select the continue button 818 to access next steps of the registration process.

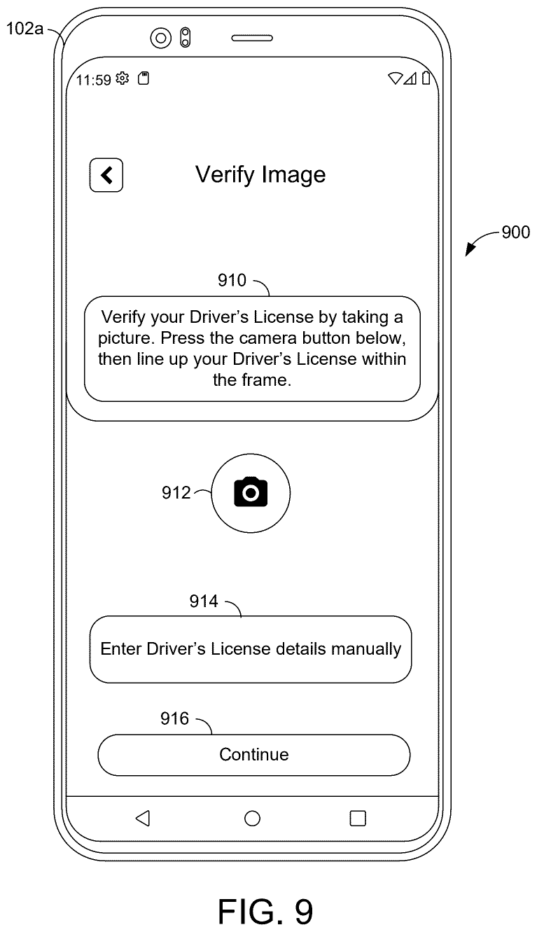

Turning now to FIG. 9, a license verification screen 900 for a medical information sharing service is shown, in accordance with an aspect of the technology described herein. The license verification screen 900 is displayed on a user device 102a. The license verification screen 900 uses a potential user's driver's license to further confirm the identity of the potential user. Instead of a driver's license, other government ID that includes a picture may be provided. The license verification screen 900 provides verification instructions 910 to the user. The instructions ask the user to line up their driver's license in a frame that will be displayed within the user interface. When properly framed, the user may take a picture of the driver license by pushing the picture button 912. To use the camera features of the user device, the medical information sharing service may seek permission from the user for camera access. This may include the user going to device settings and granting access to the medical-information application. In addition to or alternative to the picture of the ID, the user may select the manual enter button 914 to manually enter driver's license details, such as home address, driver's license number, date of birth, etc. Selecting the continue button 916 accesses next steps of the registration process. At this point, the medical information sharing service may compare the picture of the government ID with a live image of the user. If the live image matches the image on the government ID, then the ID may be considered validated. The medical information sharing service may then associate a facial profile with the user account. This may be used subsequently to login and possibly during system use to confirm the medical information is being presented to the authorized user.

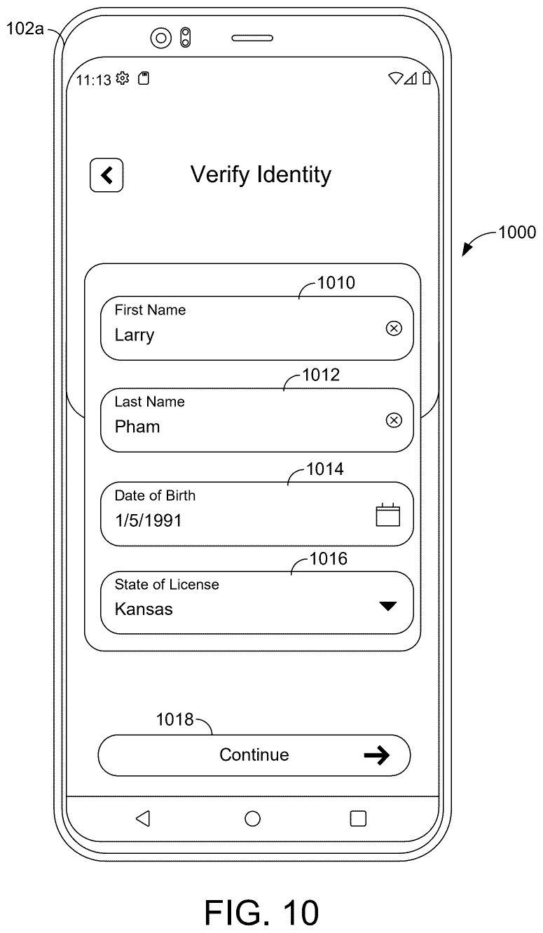

Turning now to FIG. 10, a manual identity verification screen 1000 for a medical information sharing service is shown, in accordance with an aspect of the technology described herein. The manual identity verification screen 1000 is displayed on a user device 102a. The manual identity verification screen 1000 allows the user to enter details from their driver's license, medical license, or other source. The manual identity verification screen 1000 includes a first name text box 1010, a last name text box 1012, a date of birth text box 1014, and a state of license text box 1016. The user may enter the requested information in the various boxes and press the continue button 1018 to access the next steps of the registration process.

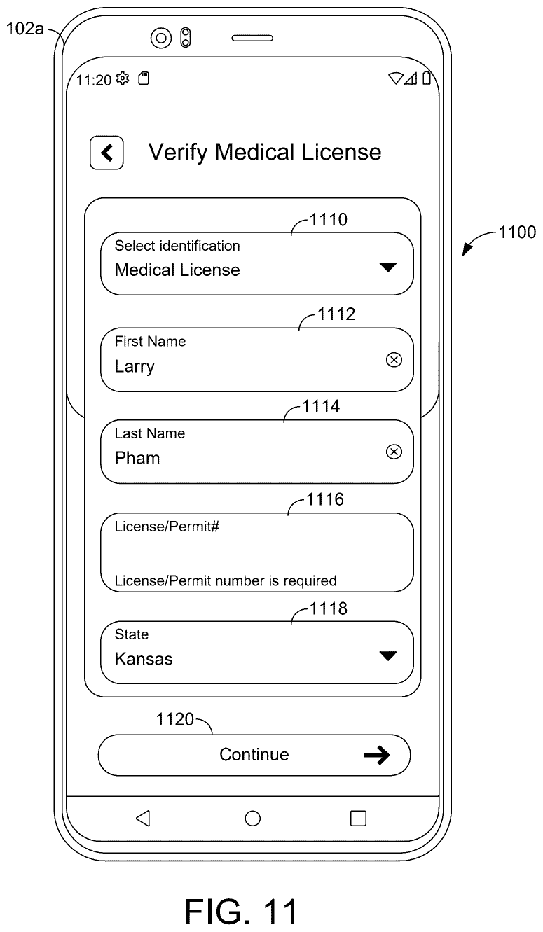

Turning now to FIG. 11, a medical license verification screen 1100 for a medical information sharing service is shown, in accordance with an aspect of the technology described herein. The medical license verification screen 1100 is displayed on a user device 102a. As described previously, the purpose of the medical information application is to limit access to medical professionals who are authorized to share and receive medical information. The medical license verification 1100 screen includes an identification type selection box 1110 through which a user can designate the type of credential to be validated. In this case a medical license, such as issued by U.S. states, is selected. The user may enter a first name in first name box 1112, a last name in the last name box 1114, a medical license number in the number box 1116, and a state of licensure in the state box 1118. Pressing the continue button 1120 accesses the next screen in the verification process. The medical information sharing service may then compare the information provided with a medical provider database. If the information matches, then the account may be set up for the user. The potential user then becomes an authorized user.

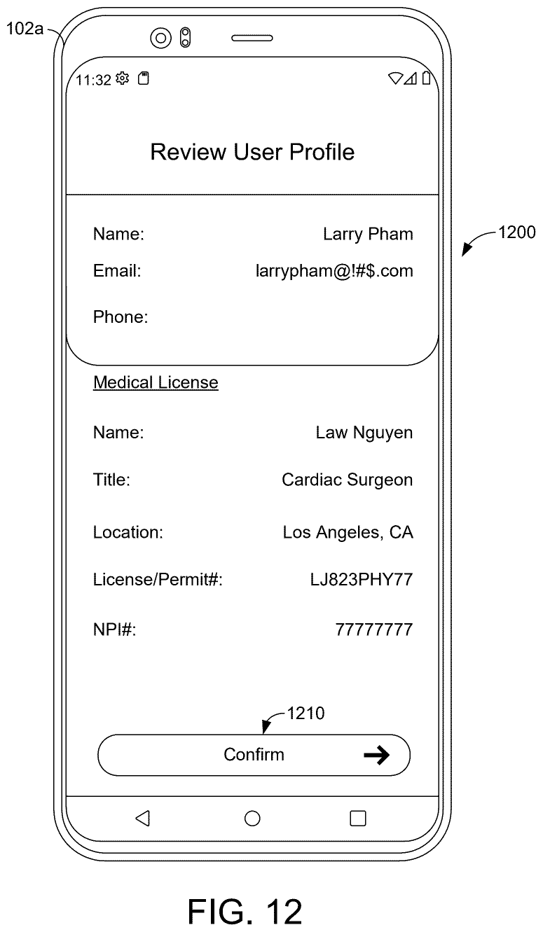

Turning now to FIG. 12, a registration review screen 1200 for a medical information sharing service is shown, in accordance with an aspect of the technology described herein. The registration review screen 1200 is displayed on a user device 102a. The registration review screen 1200 displays information submitted during the registration process. The user may confirm that the data is accurate by selecting the confirm button 1210. If validation of information previously provided has not already been validated by various services, then it may be validated at this time.

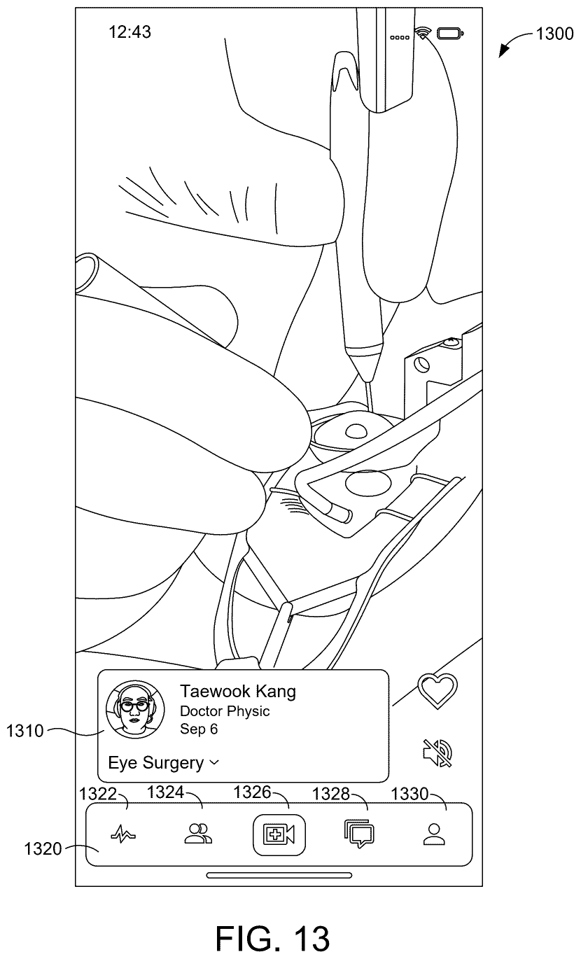

Turning now to FIG. 13, a video view screen 1300 for a medical information sharing service is shown, in accordance with an aspect of the technology described herein. The video view screen 1300 is displayed on a user device 102a. Videos of medical procedures are one type of medical information that may be shared through the medical information sharing service. The video view screen 1300 illustrates the display of a video showing a doctor performing an eye surgery. The caption 1310 identifies the user posting the video along with the caption eye surgery. The navigation bar 1320 allows the user to navigate to other content related to the video. For example, a first icon 1322, may provide additional information related to the category assigned to the video. For example, additional videos or content related to eye surgery may be found by selecting the first icon. A colleague icon 1324 enables a user to view connections within the medical sharing service and identify additional connections. A connection may be another user the viewer has an established relationship with within the service. For example, the viewer may follow multiple content creators. Additional connections may be suggested based on common relationships, common interests, or other characteristics in common. The video icon 1326 is highlighted and indicates the current view. The chat icon 1328 allows the user view and make posts about the video. The people icon 1330 may indicate other people watching the video.

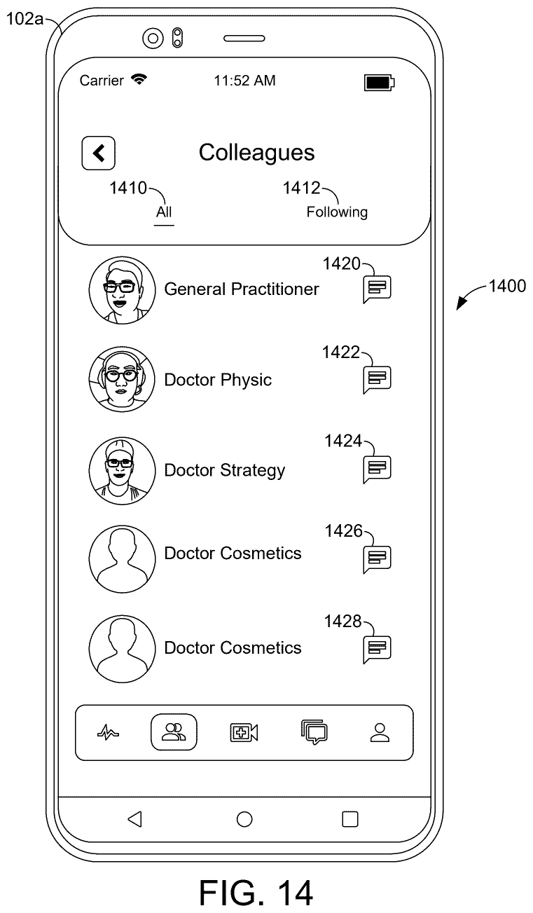

Turning now to FIG. 14, a medical professional network 1400 screen for a medical information sharing service is shown, in accordance with an aspect of the technology described herein. The medical professional network 1400 is displayed on a user device 102a. The colleagues interface helps the user identify relevant content creators. The content creators are identified by name and practice. When the “all” tab 1410 is shown all content creators may be accessed. The following tab 1412 shows content creators that the user has followed. The user may follow a content creator by selecting an associated follow button. The first follow button 1420 is associated with the first content creator, the second follow button 1422 is associated with the second content creator, the third follow button 1424 is associated with the third content creator, the fourth follow button 1426 is associated with the fourth content creator, the fifth follow button 1428 is associated with the fifth content creator. Upon selecting a follow button the user may automatically be allowed to follow the content creator. In other aspects, the content creator may need to authorize the user to follow the content creator.

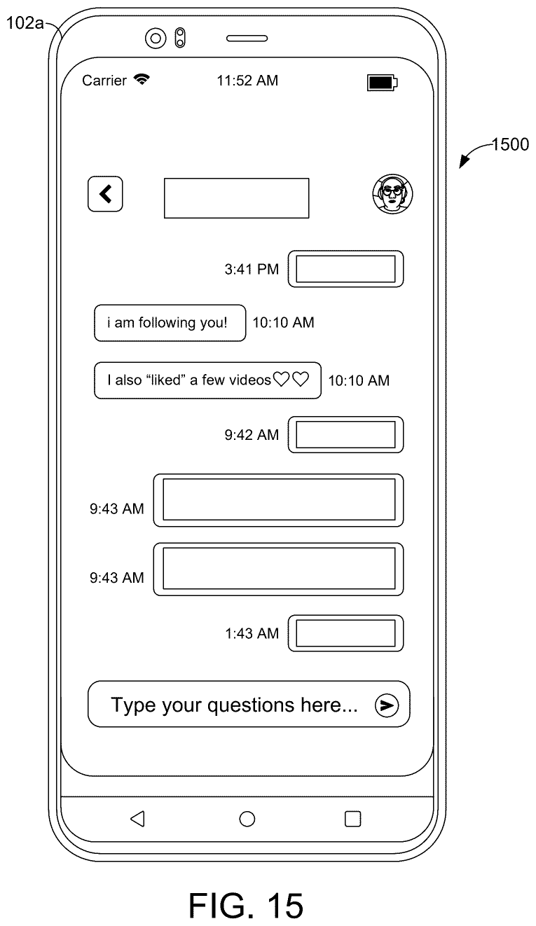

Turning now to FIG. 15, an instant message screen 1500 for a medical information sharing service is shown, in accordance with an aspect of the technology described herein. The instant message screen 1500 is displayed on a user device 102a. The instant message screen 1500 allows users to communicate with each other. The messages may be associated with a particular content, such as a video, or may more generally be between users. When associated with content, the instant messaging may be available to other users through an interface associated with the video. In an aspect, the instant messaging may act as a comment section for a video.

Turning now to FIG. 16, a profile screen 1600 for a medical information sharing service is shown, in accordance with an aspect of the technology described herein. The profile screen 1600 is displayed on a user device 102a. The profile screen 1600 allows a user to post new content and view previously posted content. In this case, the example user has a video gallery 1610 of previously posted videos. The first video 1620 and the second video 1622 are described as inactive. An inactive video may have been uploaded to the system but made unavailable to other users. In contrast, an active video may be published and viewable within the system. A creator may publish to a select group of users or more broadly to any user within the system. The system may provide various methods of identifying content including a search engine that searches on topic, creator, date published, video title, etc.

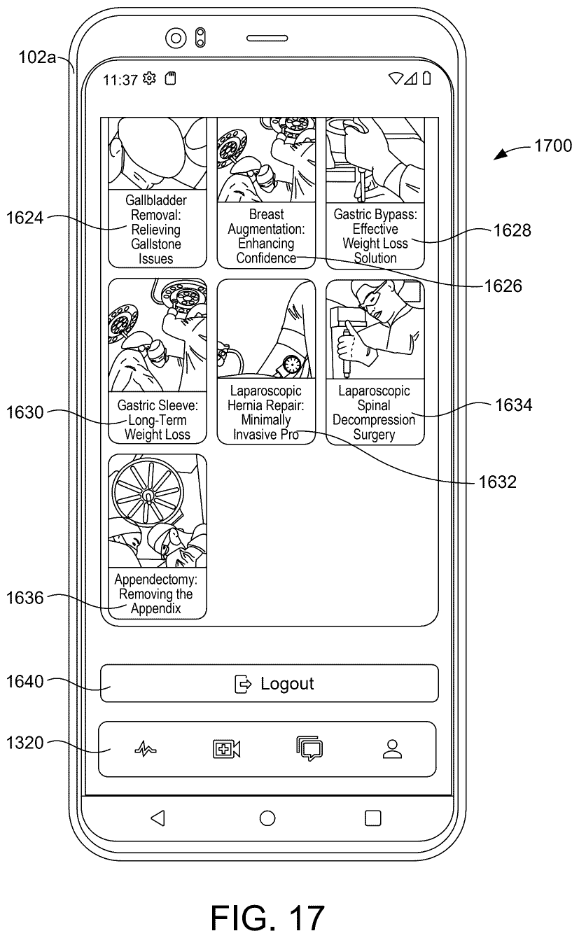

Turning now to FIG. 17, a video gallery screen 1700 for a medical information sharing service is shown, in accordance with an aspect of the technology described herein. The video gallery screen 1700 is displayed on a user device 102a. The video gallery screen 1700 shows videos previously uploaded by the user. For the sake of illustration, thumbnails for seven videos are shown. The user may access the videos by selecting the thumbnail. The first video may be accessed by selecting a first thumbnail 1624, a second video may be accessed by selecting a second thumbnail 1626, third video may be accessed by selecting a third thumbnail 1628, a fourth video may be accessed by selecting a fourth thumbnail 1630, a fifth video may be accessed by selecting a fifth thumbnail 1632, a sixth video may be accessed by selecting a sixth thumbnail 1634, and a seventh video may be accessed by selecting a seventh thumbnail 1636. A logout button 1640 facilitates logging out of the application. Upon selecting any thumbnail, various actions may be suggested. These actions can include viewing the video, deleting the video (either from the system or from a group of videos), inactivating the video, activating the video, writing a review of the video, and the like. Some actions may require system permissions. For example, content creators may be able to delete their content, but not the content of others.

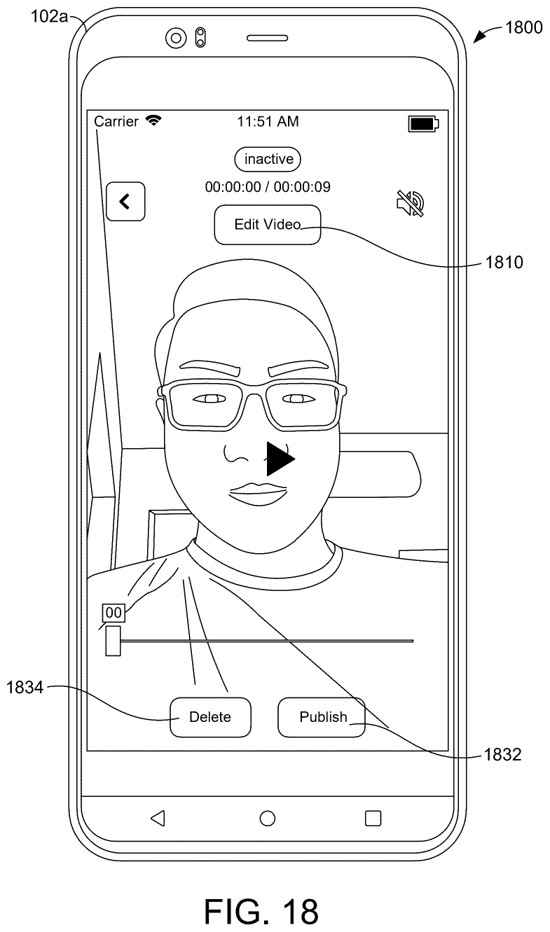

Turning now to FIG. 18, a video creation screen 1800 for a medical information sharing service is shown, in accordance with an aspect of the technology described herein. The video creation screen 1800 is displayed on a user device 102a. Upon uploading a video, a user may be given the option to edit the video by selecting the edit button 1810. Upon selecting the edit button 1810, a video editing interface may be opened. If the user is satisfied with the video the user may choose to publish it by selecting the publish button 1832. Publishing a video makes it accessible to other users on the platform. A video may be generally available to all authorized users or be available to a more limited group. For example, a video of eye surgery may be made available to doctors that have signed up for a particular refresher course associated with the video. In another aspect, videos are available to followers of a user that published the video. A user may put a policy in place to determine which other users may follow his or her content. If the user is unsatisfied with the video, the video may be deleted by pushing the delete button 1834.

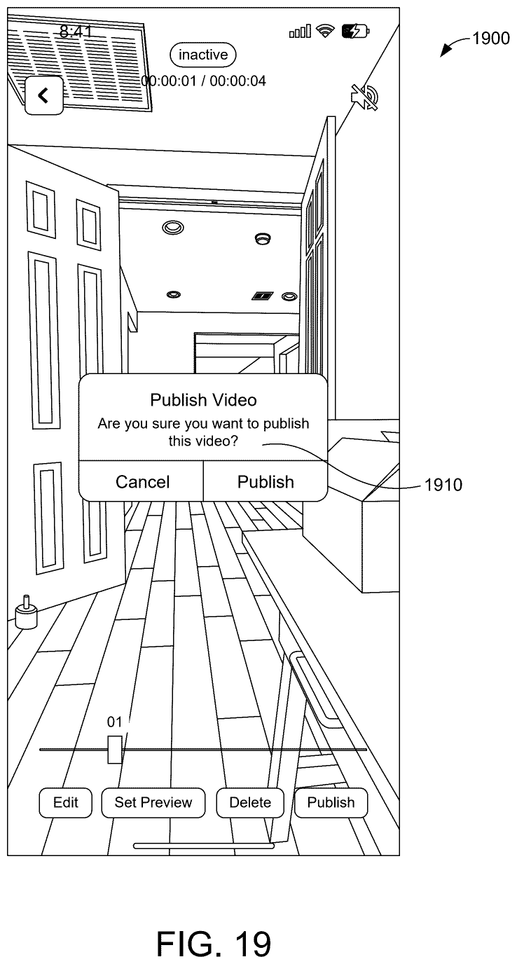

Turning now to FIG. 19, a video publication screen 1900 for a medical information sharing service is shown, in accordance with an aspect of the technology described herein. The video publication screen 1900 is displayed on a user device 102a. The video publication screen 1900 asks the user to confirm that a video should be published by selecting the publish button 1910. The video publication screen 1900 be shown in response to the user selecting the publish button 1832.

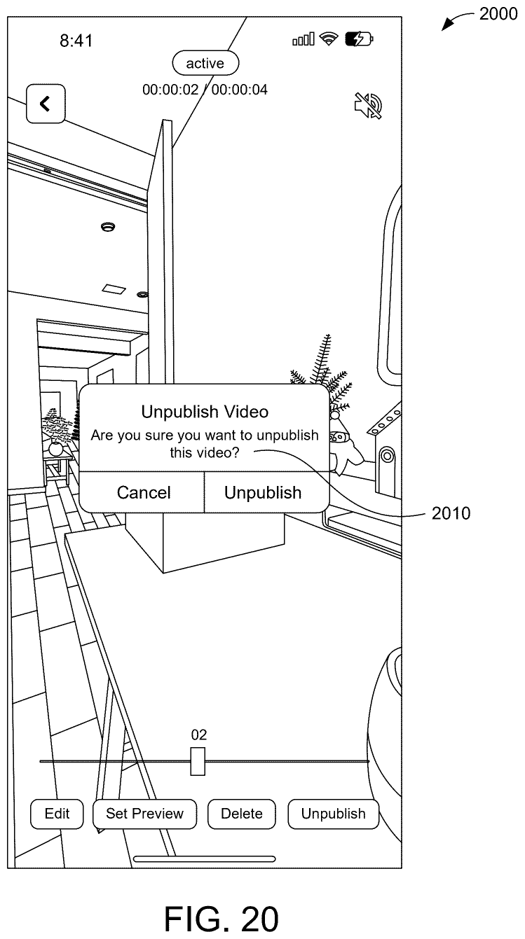

Turning now to FIG. 20, a video removal screen 2000 for a medical information sharing service is shown, in accordance with an aspect of the technology described herein. The video removal screen 2000 is displayed on a user device 102a. The video removal screen 2000 allows a user to select one of their own videos and remove it from the platform. A user may select unpublish through the unpublish interface 2010.

### Example Operating Environment

Referring to the drawings in general, and initially to FIG. 21, an example operating environment for implementing aspects of the technology described herein is shown and designated generally as computing device 2100. Computing device 2100 is but one example of a suitable computing environment and is not intended to suggest any limitation as to the scope of use of the technology described herein. Neither should the computing device 2100 be interpreted as having any dependency or requirement relating to any one or combination of components illustrated.

The technology described herein may be described in the general context of computer code or machine-useable instructions, including computer-executable instructions such as program components, being executed by a computer or other machine, such as a personal data assistant or other handheld device. Generally, program components, including routines, programs, objects, components, data structures, and the like, refer to code that performs tasks or implements abstract data types. The technology described herein may be practiced in a variety of system configurations, including handheld devices, consumer electronics, general-purpose computers, specialty computing devices, etc. Aspects of the technology described herein may also be practiced in distributed computing environments where tasks are performed by remote-processing devices that are linked through a communications network.

With continued reference to FIG. 21, computing device 2100 includes a bus 2110 that directly or indirectly couples the following devices: memory 2112, one or more processors 2114, one or more presentation components 2116, input/output (I/O) ports 2118, I/O components 2120, and an illustrative power supply 2122. Bus 2110 represents what may be one or more busses (such as an address bus, data bus, or a combination thereof). Although the various blocks of FIG. 21 are shown with lines for the sake of clarity, delineating various components is not so clear, and metaphorically, the lines would more accurately be grey and fuzzy. For example, one may consider a presentation component such as a display device to be an I/O component. Also, processors have memory. The inventors hereof recognize that such is the nature of the art and reiterate that the diagram of FIG. 21 is merely illustrative of a computing device that may be used in connection with one or more aspects of the technology described herein. Distinction is not made between such categories as “workstation,” “server,” “laptop,” “handheld device,” etc., as all are contemplated within the scope of FIG. 21 and refer to “computer” or “computing device.”

Computing device 2100 typically includes a variety of computer-readable media. Computer-readable media may be any available media that may be accessed by computing device 2100 and includes both volatile and nonvolatile, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules, or other data.

Computer storage media includes RAM, ROM, EEPROM, flash memory or other memory technology, CD-ROM, digital versatile disks (DVD) or other optical disk storage, magnetic cassettes, magnetic tape, magnetic disk storage or other magnetic storage devices. Computer storage media does not comprise a propagated data signal.

Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media.

Memory 2112 includes computer storage media in the form of volatile and/or nonvolatile memory. The memory 2112 may be removable, non-removable, or a combination thereof. Example memory includes solid-state memory, hard drives, optical-disc drives, etc. Computing device 2100 includes one or more processors 2114 that read data from various entities such as bus 2110, memory 2112, or I/O components 2120. Presentation component(s) 2116 present data indications to a user or other device. Example presentation components 2116 include a display device, speaker, printing component, vibrating component, etc. I/O ports 2118 allow computing device 2100 to be logically coupled to other devices, including I/O components 2120, some of which may be built in.

Illustrative I/O components include a microphone, joystick, game pad, satellite dish, scanner, printer, display device, wireless device, a controller (such as a stylus, a keyboard, and a mouse), a natural user interface (NUI), and the like. In aspects, a pen digitizer (not shown) and accompanying input instrument (also not shown but which may include, by way of example only, a pen or a stylus) are provided to digitally capture freehand user input. The connection between the pen digitizer and processor(s) 2114 may be direct or via a coupling utilizing a serial port, parallel port, and/or other interface and/or system bus known in the art. Furthermore, the digitizer input component may be a component separated from an output component such as a display device, or in some aspects, the usable input area of a digitizer may coexist with the display area of a display device, be integrated with the display device, or may exist as a separate device overlaying or otherwise appended to a display device. All such variations, and any combination thereof, are contemplated to be within the scope of aspects of the technology described herein.

An NUI processes air gestures, voice, or other physiological inputs generated by a user. Appropriate NUI inputs may be interpreted as ink strokes for presentation in association with the computing device 2100. These requests may be transmitted to the appropriate network element for further processing. An NUI implements any combination of speech recognition, touch and stylus recognition, facial recognition, biometric recognition, gesture recognition both on screen and adjacent to the screen, air gestures, head and eye tracking, and touch recognition associated with displays on the computing device 2100. The computing device 2100 may be equipped with depth cameras, such as stereoscopic camera systems, infrared camera systems, RGB camera systems, and combinations of these, for gesture detection and recognition. Additionally, the computing device 2100 may be equipped with accelerometers or gyroscopes that enable detection of motion. The output of the accelerometers or gyroscopes may be provided to the display of the computing device 2100 to render immersive augmented reality or virtual reality.

A computing device may include a radio 2124. The radio 2124 transmits and receives radio communications. The computing device may be a wireless terminal adapted to receive communications and media over various wireless networks. Computing device 2100 may communicate via wireless policies, such as code division multiple access (“CDMA”), global system for mobiles (“GSM”), or time division multiple access (“TDMA”), as well as others, to communicate with other devices. The radio communications may be a short-range connection, a long-range connection, or a combination of both a short-range and a long-range wireless telecommunications connection. When we refer to “short” and “long” types of connections, we do not mean to refer to the spatial relation between two devices. Instead, we are generally referring to short range and long range as different categories, or types, of connections (i.e., a primary connection and a secondary connection). A short-range connection may include a Wi-Fi® connection to a device (e.g., mobile hotspot) that provides access to a wireless communications network, such as a WLAN connection using the 802.11 protocol. A Bluetooth connection to another computing device is a second example of a short-range connection. A long-range connection may include a connection using one or more of CDMA, GPRS, GSM, TDMA, and 802.16 policies.

### Example Methods

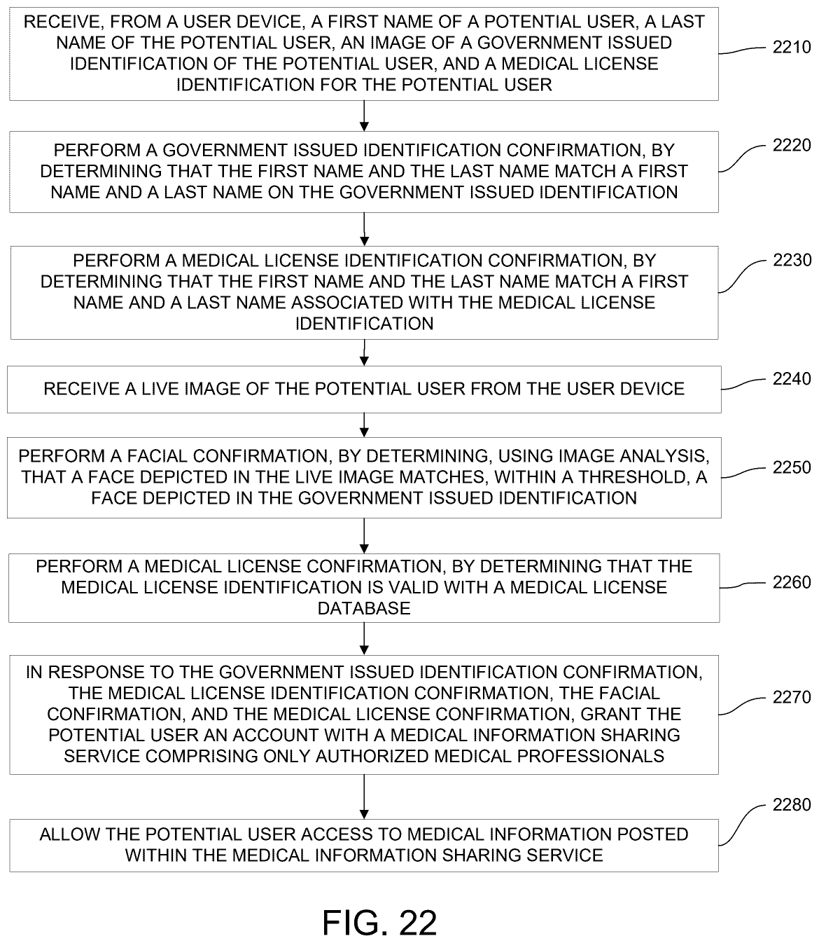

Now referring to FIGS. 22, 23 and 24, each block of methods 2200, 2300, and 2400, described herein, comprises a computing process that may be performed using any combination of hardware, firmware, and/or software. For instance, various functions may be carried out by a processor executing instructions stored in memory. The methods may also be embodied as computer-usable instructions stored on computer storage media. The method may be provided by an operating system. In addition, methods 2200, 2300, and 2400 are described, by way of example, with respect to FIGS. 1-21. However, these methods may additionally or alternatively be executed by any one system, or any combination of systems, including, but not limited to, those described herein.

FIG. 22 is a flow diagram showing a method 2200 of sharing medical information, in accordance with some embodiments of the present disclosure. Method 2200 may be performed on or with systems similar to those described with reference to FIGS. 1-21.

At step 2210, the method 2200 includes receiving, from a user device, a first name of a potential user, a last name of the potential user, an image of a government issued identification of the potential user, and a medical license identification for the potential user.

At step 2220, the method 2200 includes performing a government issued identification confirmation, by determining that the first name and the last name match a first name and a last name on the government issued identification.

At step 2230, the method 2200 includes performing a medical license identification confirmation, by determining that the first name and the last name match a first name and a last name associated with the medical license identification.

At step 2240, the method 2200 includes receiving a live image of the potential user from the user device.

At step 2250, the method 2200 includes performing a facial confirmation, by determining, using image analysis, that a face depicted in the live image matches, within a threshold, a face depicted in the government issued identification.

At step 2260, the method 2200 includes performing a medical license confirmation, by determining that the medical license identification is valid with a medical license database.

At step 2270, the method 2200 includes in response to the government issued identification confirmation, the medical license identification confirmation, the facial confirmation, and the medical license confirmation, granting the potential user an account with a medical information sharing service comprising only authorized medical professionals.

At step 2280, the method 2200 includes allowing the potential user access to medical information posted within the medical information sharing service.

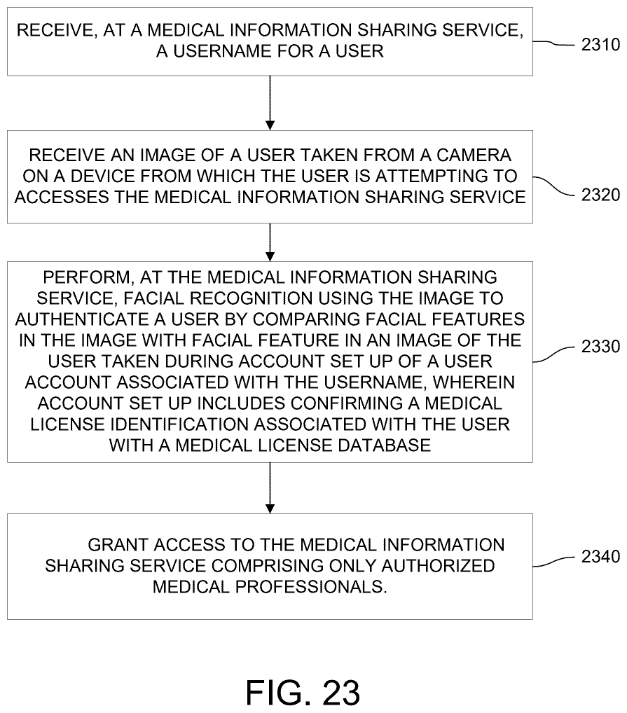

FIG. 23 is a flow diagram showing a method 2300 of sharing medical information, in accordance with some embodiments of the present disclosure. Method 2300 may be performed on or with systems similar to those described with reference to FIGS. 1-21.

At step 2310, the method 2300 includes receiving, at a medical information sharing service, a username for a user. At step 2320, the method 2300 includes receiving an image of a user taken from a camera on a device from which the user is attempting to accesses the medical information sharing service.

At step 2330, the method 2300 includes performing, at the medical information sharing service, facial recognition using the image to authenticate a user by comparing facial features in the image with facial feature in an image of the user taken during account set up of a user account associated with the username. The account set up included confirming a medical license identification associated with the user with a medical license database. At step 2340, the method 2300 includes granting access to the medical information sharing service comprising only authorized medical professionals.

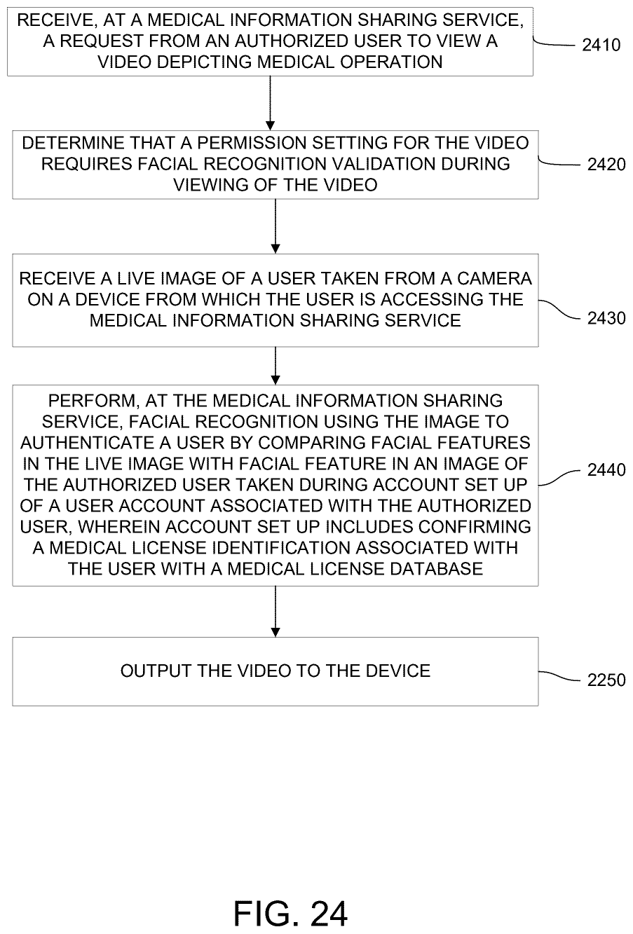

FIG. 24 is a flow diagram showing a method 2400 of sharing medical information, in accordance with some embodiments of the present disclosure. Method 2400 may be performed on or with systems similar to those described with reference to FIGS. 1-21.

At step 2410, the method 2400 includes receiving, at a medical information sharing service, a request from an authorized user to view a video depicting medical operation.

At step 2420, the method 2400 includes determining that a permission setting for the video requires facial recognition validation during viewing of the video. At step 2430, the method 2400 includes receiving a live image of a user taken from a camera on a device from which the user is accessing the medical information sharing service.

At step 2440, the method 2400 includes performing, at the medical information sharing service, facial recognition using the image to authenticate a user by comparing facial features in the live image with facial feature in an image of the authorized user taken during account set up of a user account associated with the authorized user. The account set up included confirming a medical license identification associated with the user with a medical license database. At step 2450, the method 2400 includes outputting the video to the device.

### EMBODIMENTS

The technology described herein has been described in relation to aspects, which are intended in all respects to be illustrative rather than restrictive. While the technology described herein is susceptible to various modifications and alternative constructions, certain illustrated aspects thereof are shown in the drawings and have been described above in detail. It should be understood, however, that there is no intention to limit the technology described herein to the specific forms disclosed, but on the contrary, the intention is to cover all modifications, alternative constructions, and equivalents falling within the spirit and scope of the technology described herein.

What is claimed is:

1. One or more computer storage media comprising computer-executable instructions that when executed by computing device performs a method of sharing medical information, the method comprising:
receiving, from a user device, a first name of a potential user, a last name of the potential user, an image of a government issued identification of the potential user, and a medical license identification for the potential user;
performing a government issued identification confirmation, by determining that the first name and the last name match a first name and a last name on the government issued identification;
performing a medical license identification confirmation, by determining that the first name and the last name match a first name and a last name associated with the medical license identification;
receiving a live image of the potential user from the user device;
performing a facial confirmation, by determining, using image analysis, that a face depicted in the live image matches, within a threshold, a face depicted in the government issued identification;
performing a medical license confirmation, by determining that the medical license identification is valid with a medical license database;
in response to the government issued identification confirmation, the medical license identification confirmation, the facial confirmation, and the medical license confirmation, granting the potential user an account with a medical information sharing service comprising only authorized medical professionals; and
allowing the potential user access to medical information posted within the medical information sharing service.

2. The media of claim 1, wherein the medical license database is a National Provider Identifier (NPI) database.

3. The media of claim 1, further comprising using facial recognition technology to authenticate the potential user by comparing a live image received from the user device with an image associated with the medical license.

4. The media of claim 1, wherein the medical information is selected from the group consisting of videos, images, textual descriptions, and analytical data.

5. The media of claim 1, further comprising obtaining patient consent prior to generating, broadcasting, or publishing any medical video content that depicts the patient.

6. The media of claim 1, wherein copying of videos from the medical information sharing service is prevented using digital rights management technology.

7. The media of claim 1, further comprising using native voice triggers to initiate specific camera movements during a surgical procedure to generate the medical information.

8. A method for verifying an identity of medical professionals, comprising:
receiving, at a medical information sharing service, a username for a user;
receiving an image of a user taken from a camera on a device from which the user is attempting to accesses the medical information sharing service;
performing, at the medical information sharing service, facial recognition using the image to authenticate a user by comparing facial features in the image with facial feature in an image of the user taken during account set up of a user account associated with the username, wherein account set up includes confirming a medical license identification associated with the user with a medical license database; and
granting access to the medical information sharing service comprising only authorized medical professionals.

9. The method of claim 8, wherein the facial recognition uses a live image of the user.

10. The method of claim 8, wherein the medical information sharing service comprises a messaging system that allows secure communication between authorized medical professionals.

11. The method of claim 8, wherein the medical information sharing service comprises a video capture system.

12. The method of claim 11, wherein the video capture system comprises a knowledge-based verification process to confirm an identity of a medical professional and a patient before allowing video capture of a medical procedure.

13. The method of claim 11, wherein the video capture system comprises includes an automated camera tracking system.

14. The method of claim 11, wherein the video capture system comprises a movie script integration module that synchronizes camera actions with a doctor's narration during a medical procedure.

15. A method for sharing medical information, comprising:
receiving, at a medical information sharing service, a request from an authorized user to view a video depicting medical operation;
determining that a permission setting for the video requires facial recognition validation during viewing of the video;
receiving a live image of a user taken from a camera on a device from which the user is accessing the medical information sharing service;
performing, at the medical information sharing service, facial recognition using the image to authenticate a user by comparing facial features in the live image with facial feature in an image of the authorized user taken during account set up of a user account associated with the authorized user, wherein account set up includes confirming a medical license identification associated with the user with a medical license database; and
outputting the video to the device.

16. The method of claim 15, wherein the video is a live medical procedure being broadcast in substantially real-time.

17. The method of claim 15, further comprising obtaining patient consent prior to generating, broadcasting, or publishing any medical video content that depicts the patient.

18. The method of claim 17, further comprising associating the patient consent with the medical video within a video storage system.

19. The method of claim 15, further comprising using native voice triggers to initiate specific camera movements during a surgical procedure depicted in the video.

20. The method of claim 15, further comprising a outputting messaging content from a messaging system that allows secure communication between authorized medical professional viewing the video.
